(19) United States
(12) Patent Application Publication
(10) Pub. No.: US 2009/0069812 A1
(43) Pub. Date: Mar. 12, 2009

(54) RIB FIXATION WITH AN INTRAMEDULLARY NAIL

(75) Inventors: Joel Gillard, Portland, OR (US); Randall J. Huebner, Portland, OR (US)

Correspondence Address: KOLISCH HARTWELL, P.C., 200 PACIFIC BUILDING, 520 SW YAMHILL STREET, PORTLAND, OR, 97204, US

(73) Assignee: ACUMED LLC, Hillsboro, OR (US)

(21) Appl. No.: 12/140,143

(22) Filed: Jun. 16, 2008

Related U.S. Application Data
(60) Provisional application No. 60/934,696, filed on Jun. 15, 2007.

(51) Int. Cl. A61B 17/56; A61B 19/00; A61B 17/04

(52) U.S. Cl. 606/62; 606/64; 128/898; 606/300

(57) ABSTRACT

Systems, including methods, apparatus, and kits, for fixing rib bones with a fixation device including an intramedullary nail connected to a generally U-shaped plate.

### CROSS-REFERENCE TO PRIORITY APPLICATION

This application is based upon and claims the benefit under 35 U.S.C. § 119(e) of U.S. Provisional Patent Application Ser. No. 60/934,696, filed Jun. 15, 2007, which is incorporated herein by reference in its entirety for all purposes.

### CROSS-REFERENCES TO RELATED APPLICATIONS

The following patent documents are incorporated herein by reference: U.S. Provisional Patent Application Ser. No. 60/498,866, filed Aug. 28, 2003; U.S. Provisional Patent Application Ser. No. 60/548,685, filed Feb. 26, 2004; U.S. patent application Ser. No. 10/927,824, filed Aug. 27, 2004, and published as Pub. No. U.S. 2005/0085819 A1 on Apr. 21, 2005; U.S. patent application Ser. No. 11/454,613, filed Jun. 16, 2006, and published as Pub. No. U.S. 2007/0123883 A1 on May 31, 2007; and U.K. Patent Application Serial No. 0810872.2, filed Jun. 13, 2008, titled “Rib Fixation with an Intramedullary Nail,” and naming Joel Gillard and Randall J. Huebner as inventors.

### BACKGROUND

The human skeleton is composed of 206 individual bones that perform a variety of important functions, including support, movement, protection, storage of minerals, and formation of blood cells. These bones can be grouped into two categories, the axial skeleton and the appendicular skeleton. The axial skeleton consists of 80 bones that make up the body's center of gravity, and the appendicular skeleton consists of 126 bones that make up the body's appendages. The axial skeleton includes the skull, vertebral column, ribs, and sternum, among others, and the appendicular skeleton includes the long bones of the upper and lower limbs, and the clavicles and other bones that attach these long bones to the axial skeleton, among others.

To ensure that the skeleton retains its ability to perform its important functions, and to reduce pain and disfigurement, fractured bones should be repaired promptly and properly. Typically, fractured bones are treated using fixation devices, which reinforce the fractured bones and keep them aligned during healing. Fixation devices may take a variety of forms, including casts for external fixation and bone plates for internal fixation, among others. Casts are minimally invasive, allowing reduction and fixation of simple fractures from outside the body. In contrast, bone plates are internal devices that mount under the skin of a plate recipient and in engagement with bone to span a fracture.

Trauma to the torso may result in fracture of one or more ribs. Frequently, a simple rib fracture is nondisplaced, so that reduction and/or internal fixation of the fractured rib may not be required. However, in cases of more severe trauma to the chest, a single rib may be fractured more severely and/or multiple rib fractures may occur. With multiple rib fractures, a section of the thoracic wall may become detached from the rest of the chest wall, a condition known to medical practitioners as “flail chest.” Flail chest often results in paradoxical motion of the injured area, in which the freely floating thoracic section is drawn in during inspiration, and pushed out during expiration. This condition may result in severe respiratory distress, possibly requiring the patient to be sedated and/or intubated during early stages of healing. Fixing single or multiple rib fractures internally may alleviate paradoxical motion, reduce pain, and/or help to prevent secondary injuries.

Internal fixation of a rib fracture may be accomplished using a bone plate to span the fracture. A bone plate suitable for treating a fractured rib may be custom-contoured (i.e., bent) by a surgeon to conform to a region of a rib spanning a fracture, and then fastened to the rib on both sides of the fracture. The plate thus fixes the rib to permit healing. The plate may be fastened to the fractured rib using fasteners, such as bone screws. Alternatively, a bone plate may be used that has prongs disposed along its length. The prongs may be crimped so that they grasp the rib to fasten the bone plate to the rib.

Each of these plating techniques may have disadvantages for rib fixation. Installation of a bone plate for fib fixation may require a relatively long incision over a rib, in order to expose a sufficient surface area of the rib for receiving the bone plate. Moreover, many or all of the ribs have segments that are covered by another bone, which makes fixation with a bone plate impractical. For example, a rib fractured near its site of articulation with a vertebra or where it extends under the scapula is difficult to fix with a bone plate. In either case, the fracture site is blocked on its outer side by another bone and thus cannot be easily accessed surgically.

### SUMMARY

The present disclosure provides systems, including methods, apparatus, and kits, for fixing rib bones with a fixation device including an intramedullary nail connected to a generally U-shaped plate.

### BRIEF DESCRIPTION OF THE DRAWINGS

FIG. 1 is a fragmentary lateral view of a fractured rib bone fixed with an exemplary fixation system that utilizes a fixation device that includes an intramedullary nail connected to a U-shaped plate, in accordance with aspects of the present disclosure.

FIG. 2 is a sectional view of the rib bone and fixation device of FIG. 1, taken generally along line 2-2 of FIG. 1 through the rib bone and the nail.

FIG. 3 is a fragmentary sectional view of the fixation device of FIG. 1, taken generally along line 3-3 of FIG. 1 through a flexible connector region of the fixation device.

FIG. 4 is a cross-sectional view of the rib bone and fixation device of FIG. 1, taken generally along line 4-4 of FIG. 1 through the rib bone, the plate, and a threaded fastener securing the plate to the rib bone.

FIG. 5 is a fragmentary sectional view of the plate and threaded fastener taken generally along line 5-5 of FIG. 4.

FIG. 6 is a fragmentary lateral view of a fractured rib bone that is being prepared to receive the nail of the fixation device of FIG. 1 during performance of an exemplary method of rib fixation illustrated by FIGS. 6-10, in accordance with aspects of the present disclosure.

FIG. 7 is a fragmentary lateral view of the fractured rib bone of FIG. 6 with the nail partially advanced into the rib bone, in accordance with aspects of present disclosure.

FIG. 8 is a fragmentary lateral view of the fractured rib bone of FIG. 6 with the nail fully advanced into the rib bone and spanning the fracture in the rib bone, in accordance with aspects of present disclosure.

FIG. 9 is a sectional view of the fractured rib bone and fixation device of FIG. 8, taken generally along line 9-9 of FIG. 8 through the plate of the fixation device with the plate inverted, in accordance with aspects of present disclosure.

FIG. 10 is a sectional view of the fractured rib bone and fixation device of FIG. 8, taken as in FIG. 9 but after the plate has been pivoted onto the rib bone and as a threaded fastener is being installed to attach the plate to the rib bone, in accordance with aspects of present disclosure.

FIG. 11 is a fragmentary superior view of a fractured rib bone articulating with a thoracic vertebra and fixed in a posterior segment of the rib bone using another exemplary fixation device that includes an intramedullary nail connected to a U-shaped plate, with the intramedullary nail extending from a central region of the U-shaped plate, in accordance with aspects of the present disclosure.

FIG. 12 is a fragmentary posterior view of the rib bone and fixation device of FIG. 11 taken in the absence of the thoracic vertebra.

FIG. 13 is a fragmentary lateral view of a fractured rib bone fixed with yet another exemplary fixation device that includes an intramedullary nail connected to a U-shaped plate, with the nail and plate being formed by discrete nail and plate pieces, in accordance with aspects of the present disclosure.

FIG. 14 is a side view of the nail piece of the fixation device of FIG. 13.

FIG. 15 is a top view of the nail piece of the fixation device of FIG. 13.

FIG. 16 is a sectional view of the nail piece of FIG. 14, taken generally along line 16-16 of FIG. 14.

FIG. 17 is a sectional view of another exemplary nail piece, taken generally as in FIG. 16, in accordance with aspects of present disclosure.

FIG. 18 is a cross-sectional view of the rib bone and fixation device of FIG. 13, taken generally along line 18-18 of FIG. 13 through the rib bone, the discrete pieces forming the plate and the nail, and a threaded fastener connecting the discrete pieces.

FIG. 19 is a fragmentary lateral view of a fractured rib bone fixed with an exemplary fixation device that includes an intramedullary nail connected to the rib bone with a tether, in accordance with aspects of the present disclosure.

FIG. 20 is a sectional view of the rib bone and fixation device of FIG. 19, taken generally along line 20-20 of FIG. 19 through the rib bone and the nail.

FIG. 21 is a fragmentary side view of still yet another exemplary fixation device for rib fixation that includes an intramedullary nail connected to a U-shaped plate, with the device including a flexible connector region with opposing slits, in accordance with aspects of the present disclosure.

### DETAILED DESCRIPTION

The present disclosure provides systems, including methods, apparatus, and kits, for fixing rib bones with a fixation device including an intramedullary nail (also termed an intramedullary rod) connected (or connectable) to a generally U-shaped plate.

To perform a method of fixing a rib bone, a nail member may be placed along a medullary canal of a rib bone to span a discontinuity (a fracture or cut) in the rib bone. A generally U-shaped plate member may be disposed on the rib bone with a pair of arms of the plate member disposed adjacent generally opposing sides of the rib bone, such as respective inner and outer surface regions of the rib bone. The plate member may be attached to the rib bone with at least one fastener, which may extend into and/or through at least one aperture of the plate member and into and/or through the rib bone. In some cases, the fastener may extend to each member of a pair of aligned apertures defined by the pair of arms. The fastener may be a threaded fastener that locks to (achieves a threaded engagement with) the at least one aperture and/or with one or both members of the pair of aligned apertures. The nail member may have a connection to the plate member, at least after, if not before, the nail and plate members are fully installed respectively in and on the rib bone, such that the plate member attached to the rib bone restricts the nail member from migrating in the rib bone, such as to restrict the nail member from backing out of the rib bone. The nail member and the plate member may have a connection to one another before the nail member and the plate member are positioned respectively in and on the rib bone. For example, the nail member and the plate member may be unitary with one another (i.e., provided collectively by one-piece construction), if the members have a connection before installation, or may be provided by discrete nail and plate pieces that are configured to be connected to another before or after the nail and plate pieces are positioned with respect to the rib bone, but typically during installation.

The fixation device may include any suitable structure to facilitate its function and/or installation. For example, the arms of the plate member may be connected by a bridge region, and the nail member may be connected to the plate member centrally along the U-shape defined by the plate member, that is, connected via the bridge region. With this configuration, the nail member may be placed into a rib bone from a superior side of the rib bone. Alternatively, or in addition, the fixation device may include a connector region disposed between the nail member and the plate member, at least after, if not before, the members are fully installed in and on the rib bone. The connector region may be configured to be more flexible than the nail member and/or the plate member such that the fixation device can be deformed selectively in the connector region to change a relative disposition of the nail member and the plate member, which may facilitate placement of the plate member onto the rib bone, with the nail member in the rib bone, and/or may facilitate alignment of nail and plate pieces, among others.

The fixation device and methods of use disclosed herein may solve one or more problems presented by the prior art. These problems may include (1) the tendency of a K-wire or bone screw to migrate or back out of a rib bone (due to the softness and flexibility of the rib bone necessary for breathing), which can result in loss of rib fixation, (2) the inadequate strength of a K-wire for rib fixation, (3) the ability of a migrating K-wire to penetrate and migrate through soft tissue, which may even injure the heart, (4) the inaccessibility of many rib fracture sites for fixation with a bone plate, and/or (5) the relatively long incision and extent of soft tissue injury required for installation of a bone plate on a rib bone.

Further aspects of the present disclosure are described in the following sections: (I) overview of an exemplary fixation system, (II) exemplary methods of fixing a rib bone, (III) nails, (IV) plates, and (V) examples.

### I. OVERVIEW OF AN EXEMPLARY FIXATION SYSTEM

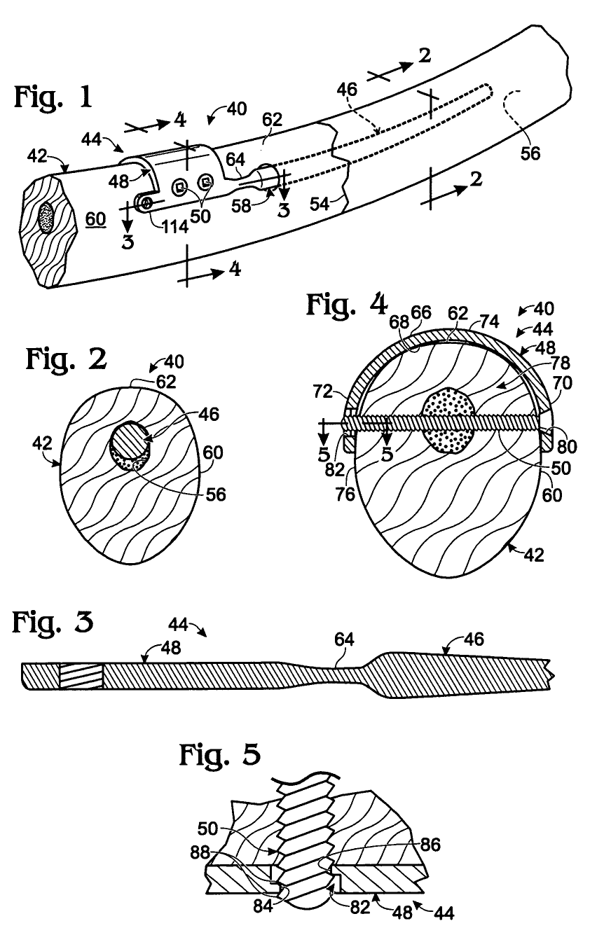

FIG. 1 shows an exemplary fixation system 40 for fixing a fractured rib bone 42, such as a human rib bone. System 40 may utilize a fixation device 44 comprising a nail 46 (also termed a nail member, a nail portion, a rod, or a rod portion) connected to a generally U-shaped plate 48 (also termed a plate member, a U-plate, a plate portion, a clip portion, or a hook portion). The nail and plate may be connected integrally (i.e., unitarily) as one piece, such as being formed by the same monolithic structure, as shown here, or may be provided by discrete pieces that are connected by a fastener mechanism. The fixation device may be attached to rib bone 42 using one or more fasteners, such as threaded fasteners 50 (i.e., bone screws) received in apertures of plate 48. Alternative mechanisms for securing the plate using an integral or discrete tie member, such as a suture or cable tie, among others, are described in U.S. Provisional Patent Application Ser. No. 60/934,696, filed Jun. 15, 2007, which is incorporated herein by reference.

Rib bone 42 may include at least one discontinuity (i.e., a fracture 54 or a cut) that is spanned by nail 46 inside the rib bone. The nail may be configured to be disposed longitudinally in the rib bone such that the nail extends along a medullary canal 56 of the rib bone. Any suitable portion or all of the nail may extend along the medullary canal. The nail may enter the rib bone at an entry site 58 on any suitable side or surface region of the rib bone, such as an outer side 60 or a superior side 62, among others.

FIG. 2 shows a sectional view of system 40 taken generally along line 2-2 of FIG. 1 through rib bone 42 and nail 46. The nail may be round-shaped in cross section, as shown here, to allow the nail to be pivoted more readily about its long axis. Alternatively, the nail may have a noncircular cross section to restrict the nail from turning in the bone. In any event, the nail may be sized according to medullary canal 56. Here, and in the other drawings of the present disclosure, the medullary canal is shown schematically and not to scale. In particular, the medullary canal may be substantially larger than shown here (relative to the transverse size of the rib bone), for example, representing at least about one-half of the thickness and at least about one-half of the width of a rib bone. In exemplary embodiments, the human medullary canal may be about 3-5 millimeters in thickness and about 3-8 millimeters in width, and may have a generally circular, oval, or ovoid cross-sectional shape, among others. Accordingly, the nail may have a transverse dimension, relative to the thickness/width of the rib bone, that is much greater than that shown in the drawings.

FIG. 3 shows a fragmentary sectional view of fixation device 44, taken generally along line 3-3 of FIG. 1. Device 44 may have a connector region 64 disposed between nail 46 and plate 48, which may form at least part of a connection of the nail to the plate before and/or after the nail and plate are installed respectively in and on the rib bone. Connector region 64 may be configured to be more flexible than nail 46 (and, optionally, plate 48). For example, connector region 64 may be more slender than the nail, such as narrower (see FIG. 1) and/or thinner. A flexible connector region between nail 46 and plate 48 permits the relative disposition of nail 46 and plate 48 to be adjusted before and/or during installation of the fixation device. In some examples, the relative disposition may be adjusted with nail 46 at least partly (or at least substantially fully) disposed in the rib bone.

FIG. 4 shows a cross-sectional view of rib bone 42 and fixation device 44, taken generally along line 4-4 of FIG. 1 through the rib bone, plate 48, and threaded fastener 50. Plate 48 may have a U-shape when viewed from the end, that is, when viewed generally parallel to an outer face 66 and an inner face 68 of the plate. The U-shape may be formed by a pair of arms 70, 72 connected by a bridge region 74 that forms a base region of the U-shape (when the plate is inverted relative to the position of FIG. 4). Arms 70, 72 may be disposed adjacent respective outer and inner surface regions 60, 76 of the rib bone, and bridge region 74 may be disposed adjacent superior surface region 62. Plate 48 thus may define a central passage 78 bounded on three sides by the bridge region and the arms of the plate. The central passage may be sized to receive a portion of rib bone 42, such as an upper portion of the rib bone when plate 48 is advanced onto the rib bone from above (superior to) the rib bone.

Arms 70, 72 each may define a member of a pair of aligned apertures, namely, an outer aperture 80 and an inner aperture 82. Threaded fastener 50 may extend through rib bone 42 between apertures 80, 82 and may lock to at least one of the apertures, such as inner aperture 82. Accordingly, inner aperture 82 may include an internal thread. Alternatively, or in addition, inner aperture 82 may be elongate (i.e., a locking slot) as shown here.

FIG. 5 shows a sectional view of inner aperture 82 in threaded engagement with fastener 50. Inner aperture 82 may include at least one lip (e.g., opposing lips 84, 86) that is sized to be received between a pair of adjacent thread segments 88 of an external thread of fastener 50. Each lip may be linear and may extend parallel to the long axis of inner aperture 82.

Further aspects of plates and locking slots that may be suitable for the fixation devices disclosed herein are described in U.S. Provisional Patent Application Ser. No. 60/934,696, filed Jun. 15, 2007, which is incorporated herein by reference.

The plate and/or the nail of a fixation device may be formed of a biocompatible and/or bioresorbable material(s). Exemplary biocompatible materials that may be suitable include (1) metals (for example, titanium or titanium alloys, alloys with cobalt and chromium (cobalt-chrome), stainless steel, etc.); (2) plastics (for example, ultra-high molecular weight polyethylene (UHMWPE), polymethylmethacrylate (PMMA), polytetrafluoroethylene (PTFE), polyetheretherketone (PEEK), and/or PMMA/polyhydroxyethylmethacrylate (PHEMA)); and/or (3) bioresorbable (bioabsorbable) materials or polymers (for example, polymers of α-hydroxy carboxylic acids (e.g., polylactic acid (such as PLLA, PDLLA, and/or PDLA), polyglycolic acid, lactide/glycolide copolymers, etc.), polydioxanones, etc.). If provided as discrete pieces, the nail and the plate may be formed of the same material or of distinct materials.

### II. EXEMPLARY METHODS OF FIXING A RIB BONE

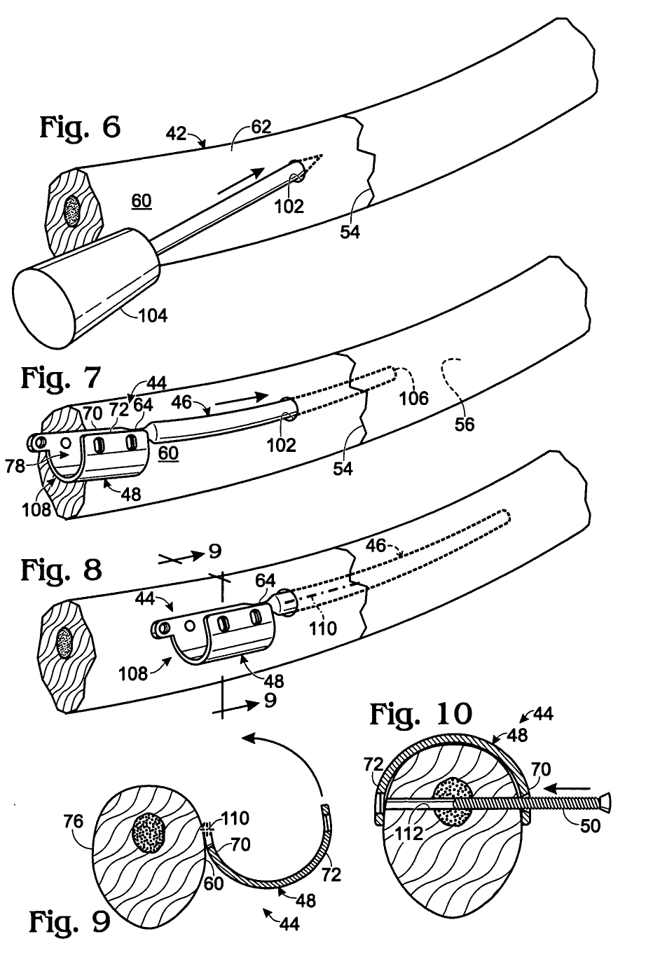

This section describes exemplary methods of fixing a rib bone with an intramedullary nail. The method steps described herein and elsewhere in the present disclosure may be performed in any suitable combination and in any suitable order. To aid in understanding the method steps, FIGS. 6-10 shows a fragmentary lateral view of fractured rib bone 42 in configurations produced during performance of an exemplary method of rib fixation with fixation device 44.

A rib bone may be selected for fixation. The rib bone may have a discontinuity (such as at least one fracture and/or a cut, among others) sustained or produced by any suitable injury or procedure. The discontinuity may be disposed medially, laterally, posteriorly, and/or anteriorly on the rib bone. In some examples, the rib bone may have sustained a fracture that is positioned inward of and thus covered by another bone, such as a vertebra or a scapula, among others.

A discontinuity in the rib bone may be reduced. For example, a fractured rib bone may be set. Reduction of the discontinuity may be performed before, during, and/or after a fixation device is attached to the rib bone, but typically before.

An aspect of the rib bone may be measured, generally in the vicinity of the discontinuity. Measurement may be performed with any suitable measuring device or method, such as calipers, a ruler, a tape measure, a fluoroscope (e.g., by fluorography), and/or the like, or any combination thereof, among others. The aspect may correspond to a characteristic dimension of the rib bone, such as thickness, width, length, diameter, or any combination thereof, among others. Alternatively, or in addition, the aspect may correspond to a curvature and/or surface contour of the rib bone, among others.

A fixation device may be selected. The fixation device may include a plate and a nail. The plate and nail may be provided by one piece or may be provided by two or more discrete pieces. The fixation device may be selected from a set of available fixation devices and/or device pieces. For example, the set may include U-shaped plates with different arm-to-arm spacings and/or radii of curvature. In other examples, the set may include nails of different length, diameter, curvature, site of connection to the plate, or any combination thereof, among others. Selection may be performed based on the type, size, and/or contour of the rib bone, among others, and thus may be based on a measured, average, and/or expected aspect (such as thickness) of the rib bone. The nail and/or plate may be manufactured with a predefined size and shape and/or may be custom contoured prior to and/or during installation for a particular rib bone, rib bone region, and/or for the particular anatomy of the patient. Custom contouring (generally, bending) may be performed pre- and/or intraoperatively by hand, with the aid of a tool, and/or with a die, among others.

FIG. 6 shows fractured rib bone 42 that has been selected and prepared to receive the nail of a fixation device. An opening 102 to the medullary canal of the rib bone may be formed using an awl 104 or other suitable hole-forming instrument. In other examples, the rib bone may be drilled, reamed, and/or broached to form a hole corresponding to the length and/or diameter of the nail. In some embodiments, the leading end of the nail may be sufficiently pointed to function as an awl. Opening 102 may be positioned to either side of fracture 54 (e.g., medially, laterally, anteriorly, and/or posteriorly to the fracture). The opening may be formed at any suitable distance from the fracture such that the distance is less than the length of the nail, for example, a distance that is about one-half the length of the nail. In addition, opening 102 may be positioned on any suitable side or surface region of the rib bone, such as outer side 60 and/or superior side 62 (see Example 1), among others. The terms outer and inner, as used herein with respect to the sides and/or surface regions of a rib bone, indicate side and/or surface regions that face generally away from (“outer”) or generally toward (“inner”) the central body axis of a patient with the rib bone.

Nail 46 of fixation device 44 may be placed into the rib bone at least partly along the medullary canal. FIG. 7 shows nail 46 received in opening 102 from the nail's leading end 106 and partially advanced longitudinally into the rib bone along medullary canal 56. FIG. 8 shows nail 46 fully advanced longitudinally into the rib bone. Placement of the nail may be facilitated by a hammer or other tool for applying an axial force to the nail. In some embodiments, the nail may include an external thread so that the nail may be driven into bone by turning the nail.

The fixation device may be installed as separate pieces or as a one-piece unit. In the case of device 44, nail 46 is already connected to plate 48 as nail 46 is advanced into the rib bone. However, if installed as a unit, the plate of the device may interfere with nail advancement and/or seating of the plate on the rib bone because of premature engagement of the plate with the rib bone. For example, with fixation device 44, if nail 46 is advanced with arms 70, 72 of plate 48 pointing inferiorly, inner arm 72 may be positioned adjacent the outer side 60 of the rib bone and through engagement with the outer side may prevent the nail from being advanced fully. Accordingly, as shown in FIGS. 8 and 9, plate 48 may be disposed an inverted orientation, indicated at 108, with passage 78 of plate 48 opening in a superior direction, during placement of the nail into the rib bone. In this inverted orientation, inner arm 72 may be generally outward of the rib bone and farther from the rib bone than outer arm 70.

The plate may be disposed on the rib bone with a pair of arms of the plate disposed adjacent respective outer and inner surface regions 60, 76 of the rib bone. Disposing the plate may include disposing the bridge region adjacent a superior or inferior surface region of the rib bone. However, placement of the bridge region superiorly may be preferable to avoid the neurovascular bundle disposed inferiorly of a rib bone. In any event, as shown in FIGS. 8 and 9, plate 48 may be pivoted about a pivot axis 110 defined by nail 46 to dispose the plate on the rib bone with arms 70, 72 opposingly flanking the rib bone. FIG. 10 shows plate 48 after the plate has been pivoted and during attachment to the rib bone with threaded fastener 50. Threaded fastener 50 may be advanced from outer arm 70, through a hole 112 formed in the rib bone, to inner arm 72. A plate with relatively shorter arms, such as extending to only about one-half or less of the width of the rib bone, may be preferable to facilitate pivoting the plate onto the rib bone. In other embodiments, the plate may be disposed on the rib bone by advancing a connected nail longitudinally into the rib bone (e.g., see Example 1) or the plate may be disposed on the rib bone as a piece separate from the nail (e.g., see Example 2).

The shape of the fixation device and the relative disposition of the nail and the plate may be adjusted at any suitable time by deforming a connector region that connects or will connect the nail and the plate to one another. The connector region thus may facilitate pivoting the plate onto the rib bone, may permit adjustment of the final position of the plate on the rib bone (such as to allow a plate to move inferiorly into contact with the rib bone), and/or may facilitate alignment of a nail piece with a plate piece during installation (e.g., see Example 2).

The plate may have a resiliency that promotes engagement with the rib bone and, optionally, provides attachment of the plate to the rib bone. In particular, the arms of the plate may be biased toward their original spacing by an integral plate spring formed by the plate, which may clamp the plate onto the rib bone. The ends of the arms may be urged or held apart as they are advanced onto the rib bone, and then may be allowed to spring back toward one another to engage the rib bone. In some embodiments, the plate may clamp onto bone via the integral plate spring for attachment without the use of fasteners, although one or more fasteners may be utilized to reinforce attachment. Furthermore, one or more prongs or ridges may be formed on an inner surface of the plate to promote engagement with the rib bone, such as by penetrating the rib bone, to restrict slippage of the clamped plate relative to the rib bone, particularly when the plate is used without fasteners.

If the plate is attached via a fastener(s), on or more transverse holes may be formed in the rib bone for receiving the fastener (e.g., FIG. 10 illustrates hole 112). The holes may be formed with a hole-forming device, such as a drill, a punch, and/or a self-drilling bone screw, among others. If formed with a drill, a drill stop, may be used to prevent advancing the hole-forming device too far, which may cause unnecessary tissue damage and/or may remove bone unnecessarily. The holes may be formed before or after the nail and/or the plate are disposed respectively in and on the rib bone. If formed after the plate is positioned on the bone, each hole may be formed in alignment with at least one aperture or a pair of aligned apertures of the plate. Accordingly, the aperture(s) may function, at least partially, as a guide for the hole-forming tool. Alternatively, a guide device may be temporarily mounted to the plate to guide a drill or other hole-forming device. For example, plate 48 may have a tab 114 defining a locking aperture for attachment of a guide device (see FIG. 1). Each hole may extend from a plate aperture into and/or through a rib bone. In some examples, each hole may extend between a pair of aligned, spaced apertures of the plate.

The plate may be attached to the rib bone with at least one fastener, such as a threaded fastener(s) and/or a tie mechanism(s), among others. One or more fasteners thus may be selected. The fasteners may be selected, for example, to have a diameter less than the width/diameter of a target aperture of the plate, and, if threaded, to have a thread configuration corresponding to the size/offset of an aperture lip (for an elongate locking aperture) or to the pitch of an aperture thread (for a locking aperture having an internal thread). The fasteners also or alternatively may be selected to have a length (particularly a shaft length for a threaded fastener) that corresponds to a measured or expected thickness of the rib bone. The fastener(s) may be placed through a plate aperture and into a pre-formed hole in the rib bone or may form a hole itself. The fastener may engage the plate adjacent only one side of the rib bone and/or adjacent generally opposing surfaces of the rib bone, among others. Each fastener may extend through the plate or through overlapping plate and nail pieces. In some examples, the fastener may be tightened until the arms of the plate are compressed against the rib bone.

### III. NAILS

The fixation device may include a nail configured to be placed at least partially along a medullary canal of a rib bone. The nail thus may be elongate with a linear or bent shape. If bent, the nail may have a curvature that is restricted to a plane or that extends to three dimensions. The curvature may be introduced into the nail during manufacture, intraoperatively before placement of the nail into the rib bone, and/or as the nail in placed into the rib bone. Accordingly, the nail may be sufficiently flexible such that the nail adapts to the curvature of the rib bone's medullary canal, via contact with the rib bone, as the nail is advanced along the medullary canal. In any event, the nail may be bent according to the curvature of a rib bone.

The nail may have any suitable length. For example, the nail may have a length that is at least about the same as or greater than the width of the plate defined generally parallel to the long axis of the nail. For example, the nail may be at least about one and one-half, two, three, or four times the width of the plate, among others.

The nail may have any suitable cross-sectional shape and size. For example, the nail may have a cross-sectional shape that is uniform or that varies along the nail. Suitable cross-sectional shapes may include circular, oval, polygonal, or a combination thereof, among others. Accordingly, the nail may be cylindrical or conical along at least a portion, at least most, or at least substantially all of its length. The diameter of the nail may be substantially uniform along the length of the rod or may vary, such as tapering as the rod extends toward its leading end. The nail may have any suitable surface relief structure, i.e., projections and/or recesses formed on the surface of the nail. Exemplary surface relief structure may include longitudinal ridges and/or grooves, dimples, bumps, annular ridges and/or grooves, helical ridges and/or grooves (e.g., an external thread and/or one or more drill flutes (i.e., a self-drilling nail), among others), and/or the like. The nail may have any suitable apertures, such as one or more blind holes and/or through-holes. The apertures may extend longitudinally along and/or completely through the nail, such as to provide a cannulated nail, or may extend transversely into and/or through the nail. Each aperture may be locking (e.g., with an internal thread) or may be nonlocking. The nail may have a transverse dimension (e.g., diameter, width, and/or thickness) corresponding to the diameter of the medullary canal of a rib bone. The nail thus may be about the same size as the medullary canal transversely, may be somewhat oversized in a transverse dimension to provide a tight fit in the rib bone, and/or may be somewhat undersized in a transverse dimension to facilitate sliding the nail along the medullary canal during installation. Accordingly, the nail may have a transverse dimension that is larger than that of a K-wire, namely, greater than about 1.6 millimeters. For example, the nail may have a transverse dimension that is at least about one-fourth or one-half the thickness and/or at least about one-fourth or one-half the width of a rib bone into which the nail is placed. In exemplary embodiments, the nail may be about 3-5 or at least about 2, 3, 4, or 5 millimeters in thickness and about 3-8 or at least about 3, 4, 5, or 6 millimeters in width (or diameter), among others. The nail thus may be bigger, stronger, stiffer, more resilient, more tapered, or any combination thereof relative to a K-wire to provide substantially improved fixation in comparison to a K-wire.

### IV. PLATES

The fixation devices of the present disclosure may include a generally U-shaped plate that connects to a nail. The plate may have a bridge region and arms opposingly flanking and extending away from the bridge region.

The plate may have any suitable spacing, structure, and disposition of its arms. Generally the arms may be spaced about the same as the distance between generally opposing surfaces of a target rib bone, that is, about the width, thickness, and/or diameter of the rib bone where the plate will be disposed. However, in some examples, the arms may be spaced somewhat greater than this distance, at least when the plate is first placed on bone, to facilitate placement. Alternatively, the arms may be spaced somewhat less than this distance, so that the arms of the plate, particularly distal regions of the arms spaced from the bridge region, may be urged farther apart as the plate is placed onto bone. In some cases, the plate may be resilient with a bias to return to its original configuration, such that the arms, if urged apart by bone, tend to opposingly engage the bone due to the bias. The arms may be at least substantially parallel, or may diverge or converge toward their distal ends (spaced from the bridge region). In exemplary embodiments, the arms may have a spacing that corresponds to the thickness of a rib bone.

The plate may have any suitable width. The width of the plate may be generally constant within each arm and/or within the bridge region. Alternatively the width may vary within one or both arms, between the arms, within the bridge region, or between the arms and the bridge region. For example, the arms may taper away from the bridge region. Alternatively, or in addition, the bridge region may be narrower than the arms, to facilitate bending the bridge region (e.g., to facilitate adjustment of the spacing of the arms), or the arms may be narrower than the bridge region (e.g., to facilitate adjustment by bending the arms). In some embodiments, the plate may include one or more narrowed regions, at which the plate may be bent selectively, such as within one or both arms or the bridge region, and/or at a junction between an arm and the bridge region.

The plate may have any suitable thickness. The thickness may be selected based on various considerations, such as reducing the profile of the plate on bone, providing a sufficient strength to restrict movement of the nail, bendability, providing a sufficient thickness to form opposing lips or a thread in an aperture for engaging a fastener thread, and/or the like.

A plate may have any suitable number, shape, and arrangement of apertures. The plate may have no apertures or may define one or more apertures. Each aperture may be circular or elongate, among others. Each aperture may include or lack a counterbore. The apertures may be disposed in the arms and/or the bridge region of the plate. If two or more apertures are included in a plate, the apertures may be arrayed across the width and/or along the length of one or more arms and/or the bridge region, and/or may have a staggered disposition. In some examples, one or more pairs of apertures may be aligned, that is, configured to receive the same fastener with each aperture of the pair. Apertures of an aligned pair may be disposed in the arms and/or in the bridge region and one arm, among others. Each aligned pair of apertures may have zero, only one, or two locking apertures. Apertures of an aligned pair may have the same general shape, such as oval or circular, or may have different shapes, such as oval and circular, among others. Furthermore, apertures of an aligned pair may be of generally the same size, such as about the same length and/or width, or may have different lengths and/or widths. The thickness of the bone plate adjacent the aperture(s) (in the plate and/or other portions of the plate) may be generally the same as, less than, or greater than the thickness of the plate away from the aperture(s). Plate thinning near the apertures may provide a recess for reducing the profile of fasteners placed in the apertures, and plate thickening near the apertures may reinforce the aperture.

Each aperture may be locking or nonlocking. Locking apertures generally include a retention structure to engage a fastener, such as through a thread of the fastener, and restrict axial movement of the fastener in both axial directions. The retention structure may be one or more ridges or lips formed by the wall of a locking aperture. The ridges may be generally helical, to form a thread, at least partially linear to form a locking slot, and/or the like.

The plate may have any suitable surface structure. The surface structure may be formed by an inner surface, an outer surface, and/or a side(s)/edge(s)/end(s) disposed between the inner and outer surfaces. The surface structure may include one or more projections, such as a ridge(s) or bump(s), or one or more depressions, such as a groove(s) or dimple(s). If a projection, the projection may be relatively sharp and/or pointed or may be relatively dull and/or rounded, among others. Exemplary surface structure that may be suitable includes one or more prongs or sharp ridges to engage and/or penetrate bone, one or more projections to space the body of the plate from bone, one or more grooves or notches to receive and retain a suture, and/or the like.

### V. EXAMPLES

The following examples describe selected aspects and embodiments of the present disclosure. These examples and the various features and aspects thereof are included for illustration and are not intended to define or limit the entire scope of the present disclosure.

### Example 1

### Exemplary Fixation Device with Centered Nail

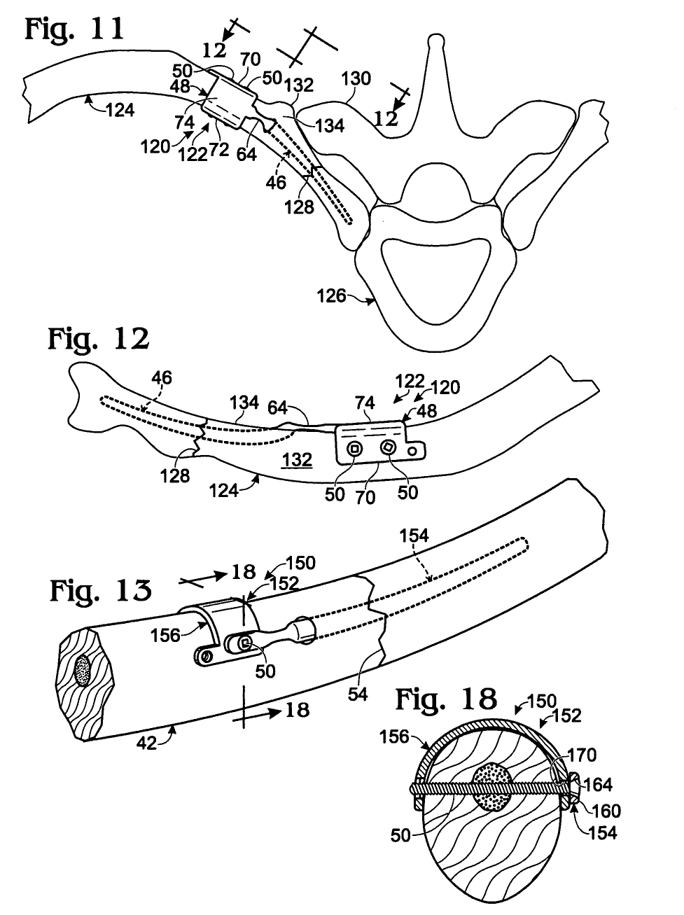

This example describes an exemplary fixation system 120 including a fixation device 122 having a nail 46 extending from a central region of U-shaped plate 48; see FIGS. 11 and 12.

Fixation device 122 may be structured for installation from a position superior (or inferior) to a rib bone (e.g., from a position between a pair of adjacent rib bones), such that nail 46 enters the rib bone from a top side or top surface region (or bottom side/surface region) of the rib bone. Device 122 may be utilized to fix any suitable rib injury but in some cases may be utilized advantageously to fix a posterior fracture of a rib bone. For example, FIG. 11 shows a fragmentary superior view of a fractured rib bone 124 articulating with a thoracic vertebra 126 and fixed in a posterior portion of the rib bone using fixation device 122. Rib bone 124 may sustain a fracture 128 that is covered on its outer side by a transverse process 130 of vertebra 126. Transverse process 130 may block or interfere with a direct posterior approach to the rib bone during surgery, for placement of a nail from an outer side 132 of the rib bone. Accordingly, nail 46 may be placed into the rib bone from the top (a posterior surface region 134) of the rib bone. FIG. 12 shows a fragmentary posterior view of rib bone 124 and fixation device 122 taken in the absence of thoracic vertebra 126.

Nail 46 and plate 48 may be structured generally as described above for fixation device 44 (see FIGS. 1-5) and/or may incorporate any of the other nail and/or plate features described elsewhere in the present disclosure. However, nail 46 may extend generally from bridge region 74 of plate 48, rather than from outer arm 70 (or inner arm) 72). In other words, nail 46 may be connected to plate 48 via bridge region 74, with or without a flexible connector region 64 disposed between the nail and the plate. The nail may be connected to the plate unitarily, such as with the nail and the plate being part of the same monolithic structure or the nail and the plate may be provided by discrete nail and plate pieces.

### Example 2

### Exemplary Fixation Device with Discrete Nail and Plate Pieces

This example describes an exemplary fixation system 150 including a fixation device 152 that includes a nail and a generally U-shaped plate formed by discrete pieces; see FIGS. 13-18.

FIG. 13 shows fractured rib bone 42 fixed with a nail piece 154 connected to a separate plate piece 156 to provide a nail member and a plate member. The nail and plate pieces may have any combination of the features described elsewhere in the present disclosure for nails and plates.

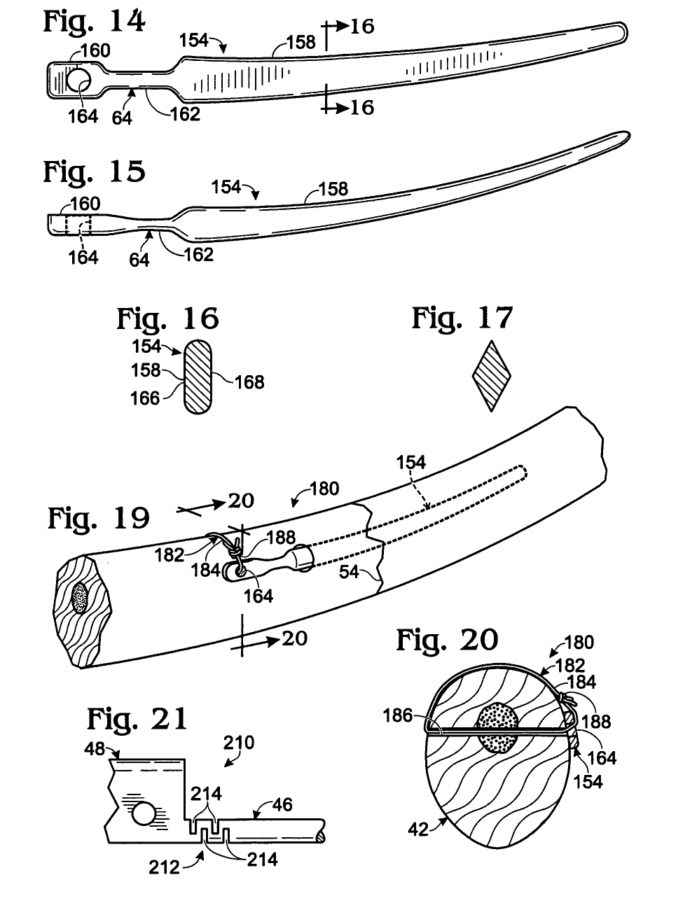

FIGS. 14 and 15 show respective side and top views of nail piece 154. The nail piece may include a body 158 connected to a head 160 by flexible connector region 64, shown here in the form of a neck 162. When installed, body 158 may be disposed inside the rib bone and head 160 may be disposed outside the rib bone. Body 158 may be linear or curved, such as with the three-dimensional curvature shown here. Head 160 may define one or more through-holes 164. If the head defines at least two through-holes, the through-holes may be spaced in correspondence with apertures of the plate piece to allow their alignment with the apertures.

FIG. 16 shows a sectional view of body 158 of nail piece 154. Body 158 may be elongate in cross section. For example, the body may have a generally flat or plate-like structure, with flat or curved opposing faces 166, 168. The cross-sectional shape thus may be generally rectangular with rounded corners, oval, or the like. FIG. 17 shows an alternative cross-sectional shape for the nail piece's body, namely, an angular cross sectional shape with a polygonal configuration, such as a square or diamond-shaped cross-section, among others.

FIG. 18 shows a sectional view of fixation system 150 taken generally along line 18-18 of FIG. 13 through the rib bone and fixation device 152. Nail piece 154 and plate piece 156 may overlap one another when connected, with the nail piece disposed farther from bone (over) or closer to bone (under) than the plate piece. For example, in FIG. 18 head 160 of the nail piece is disposed over the plate piece. Through-hole 164 of the nail piece and an aperture 170 of the plate piece may be aligned to receive threaded fastener 50. Flexible connector region 64 of the nail piece (see FIGS. 13-15) may be deformed during installation of the fixation device, such as with body 158 of the nail piece at least disposed in the rib bone, to facilitate alignment of through-hole 164 with aperture 170. Alternatively, or in addition, the entire nail piece and the entire plate piece may be moved relative to one another in and/or on the rib bone to facilitate alignment.

Fixation device 152 may be installed generally as described above in Section II. However, since the nail and plate pieces may be positioned separately, the nail piece may be placed into the rib bone at any suitable time relative to disposing the plate piece on the rib bone (i.e., before, during, and/or after).

In other embodiments, a nail piece and a plate piece may be connected to one another by other any suitable fastener mechanism. Exemplary other fastener mechanisms include threaded engagement of the nail and plate pieces with one another or attachment via a threaded fastener locks to one or both of the nail and plate pieces without extending into the rib bone. Further aspects of other suitable fastener mechanisms for connecting plate and nail pieces are described in U.S. Provisional Patent Application Ser. No. 60/934,696, filed Jun. 15, 2007, which is incorporated herein by reference.

### Example 3

### Exemplary Fixation Device with Tethered Nail

This example describes an exemplary fixation system 180 including nail piece 154 connected to a rib bone with a tether 182; see FIGS. 19 and 20.

Nail piece 154 may be placed into the rib bone and held in place with tether 182, instead of or in addition to a threaded fastener and a plate piece. The tether may be a flexible line or strap, such as a suture 184, a wire, or a cable tie (also termed a zip tie), among others. Tether 182 may be placed through through-hole 164 and also through a transverse channel 186 formed in the rib bone. The tether also may extend around at least a portion of the rib bone and may be secured to itself, such as via a knot 188, a retainer, or the like, to form a loop.

### Example 4

### Exemplary Fixation Device with Opposing Slits

This example describes an exemplary fixation device 210 with a flexible connector region created by opposing slits; see FIG. 21.

Fixation device 210 may include any of the nail and plate structure described elsewhere in the present disclosure. For example, fixation device 210 may be of one-piece construction with nail 46 connected integrally to U-plate 48 via a flexible connector region 212. The connector region may acquire at least some of its flexibility by transverse slits or openings 214 defined in the fixation device between the nail and the U-plate. The transverse slits may be defined in a plate-like or a rod-like region of the fixation device and thus may extend into the connector region from opposing sides or from three or more positions around the long axis of the nail. In any event, the transverse slits defined from generally opposing sides may be deep enough to overlap one another. In some embodiments, connector region 212 may be resilient such that slits 214 form a serpentine spring, which may flex in a plane or in three dimensions.

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure.

1. A method of fixing a rib bone, comprising:
placing a nail member in a rib bone such that the nail member extends along a medullary canal of a rib bone to span a fracture or cut in the rib bone;
disposing a generally U-shaped plate member on the rib bone with a pair of arms of the plate member disposed adjacent generally opposing surface regions of the rib bone; and
attaching the plate member to the rib bone,
wherein the nail member and the plate member have a connection to each other at least after, if not before, the steps of placing, disposing, and attaching all have been completed, such that the plate member attached to the rib bone restricts the nail member from backing out of the rib bone.

2. The method of claim 1, further comprising a step of selecting a nail member having a transverse dimension corresponding to a width or thickness of the medullary canal.

3. The method of claim 1, wherein connection of the nail member and the plate member is provided, at least in part, by a connector region disposed generally between the nail member and the plate member, wherein the connector region is more flexible than the nail member and the plate member, and wherein the step of disposing includes a step of deforming the connector region with the nail member at least partly disposed in the rib bone.

4. The method of claim 1, wherein the step of placing is performed with the nail member and the plate member connected to each other.

5. The method of claim 1, wherein the step of placing is performed at least partly with the plate member in an inverted configuration, further comprising a step of pivoting the plate member out of the inverted configuration and onto the rib bone before the step of attaching.

6. The method of claim 1, wherein the step of attaching includes a step of placing at least one threaded fastener through the rib bone between at least one pair of aligned apertures defined by the plate member such that the threaded fastener is in threaded engagement with at least one member of the pair of aligned apertures.

7. The method of claim 6, wherein the pair of aligned apertures includes a slot defined by an inner arm of the plate member, and wherein the step of placing at least one threaded fastener disposes a threaded fastener in threaded engagement with the slot.

8. The method of claim 1, wherein the plate member includes a bridge region connecting the pair of arms, and wherein the plate member has a connection to the nail member via the bridge region at least after, if not before, the steps of placing, disposing, and attaching all have been completed.

9. The method of claim 8, wherein the step of placing includes a step of inserting the nail member into the rib bone from a superior side of the rib bone.

10. The method of claim 1, wherein the nail member and the plate member are provided by a nail piece and a plate piece that are discrete from one another, and wherein the step of attaching connects the nail piece and the plate piece to teach other.

11. The method of claim 10, wherein the nail piece includes a body that forms the nail member and also includes a head connected to the body, wherein the head defines a through-hole, further comprising a step of aligning the through-hole with an aperture of the plate piece before the step of attaching by bending the nail piece between the body and head with the body disposed at least partly in the rib bone.

12. The method of claim 1, wherein the plate member is resilient to form an integral plate spring, and wherein the step of attaching includes a step of clamping the plate member onto the rib bone using the integral plate spring to urge the arms toward one another.

13. A device for fixing a rib bone, comprising:
a nail member configured to be placed in a rib bone with the nail member extending along a medullary canal of the rib bone; and
a generally U-shaped plate member connected to the nail member and including a pair of arms, the plate member being configured to be disposed on the rib bone with the pair of arms adjacent generally opposing surface regions of the rib bone.

14. The device of claim 13, wherein the nail member has a transverse dimension corresponding to a transverse size of the medullary canal.

15. The device of claim 13, further comprising a connector region disposed between the nail member and the plate member, the connector region being more flexible than the nail member and the plate member such that the connector region can be deformed selectively to change a relative disposition of the nail member and the plate member.

16. The device of claim 15, wherein the connector region is narrower than the nail member, thinner than the nail member, or both narrower and thinner than the nail member.

17. The device of claim 15, wherein the connector region defines generally opposing slits that increase the flexibility of the connector region.

18. The device of claim 13, wherein the nail member and the plate member are connected integrally as one piece.

19. The device of claim 13, wherein the nail member and the plate member are provided by a nail piece and a plate piece that are discrete from one another, wherein the nail piece and the plate piece each define an aperture, and wherein the nail piece and the plate piece are connected by a fastener disposed in the aperture of the nail member and the aperture of the plate member with the nail piece and the plate piece overlapping one another.

20. The device of claim 13, wherein the pair of arms are connected to one another in the plate member by a bridge region, and wherein the plate member is connected to the nail member via the bridge region.
